(19) United States
(12) Patent Application Publication
(10) Pub. No.: US 2022/0373536 A1
(43) Pub. Date: Nov. 24, 2022

(54) HIGH-CONTENT ANALYSIS METHOD

(71) Applicant: CELLPLY S.R.L., Bologna (IT)

(72) Inventors: Massimo BOCCHI, Bologna (IT); Andrea FAENZA, Bologna (IT); Laura ROCCHI, Bologna (IT)

(73) Assignee: CELLPLY S.R.L., Bologna (IT)

(21) Appl. No.: 17/766,154

(22) PCT Filed: Oct. 2, 2020
(86) PCT No.: PCT/IB2020/059250

§ 371 Date: Apr. 1, 2022

(30) Foreign Application Priority Data
Oct. 4, 2019 (IT) 102019000017975

(51) Int. Cl. G01N 33/50; B01L 3/00; G06V 20/69; G06T 7/00

(52) U.S. Cl. CPC G01N 33/5011; G01N 33/5047; B01L 3/5085; G06V 20/695; G06T 7/0014; G06T 2207/30096; G06V 2201/03

(57) ABSTRACT

The present invention relates to a method for subjecting a plurality of microwells containing cells to a high-content assay, said method comprising: a) Acquiring at least one image of said plurality of microwells; b) In said image, detecting a plurality of areas of interest, each area of interest corresponding to a single cell; c) Measuring at least one derived property, and, optionally, at least one direct property of said areas of interest, where said one or more properties is a selection property; d) Selecting a subset of said plurality of microwells, where said microwells belonging to the subset contain areas of interest selected based on said at least one selection property; e) Extrapolating an output parameter from a property measured in the set of areas of interest selected, where said property is defined as output property, said output property being distinct from said selection properties where said output parameter is the processing of an output property measured in said set of areas of interest. In a further aspect, there are claimed a system for subjecting a plurality of microwells containing cells to a high-content assay and a computer program which comprises instructions for subjecting a plurality of microwells containing cells to a high-content assay.

### BACKGROUND ART

Functional test-based precision medicine is an emerging field with rapidly evolving technologies.

To date, personalization through predictive functional tests is mainly linked to in-vitro analysis of the behavior of specific cell populations, such as tumor cells, analyzing parameters such as viability, immunophenotype and variation thereof following in-vitro stimulation with drugs. The cells may be cells from samples obtained from patients.

The need to obtain a datum with high predictive accuracy, necessary for the purposes of personalized medicine applications, creates the need to reproduce in-vitro conditions which allow mimicking cell interactions and the microenvironment which surrounds tumor cells in the body.

When used to assess cell death in the presence and/or absence of a therapeutic agent, flow cytometry does not allow for the collection of information on the microenvironment and cell-cell interactions.

Therefore, the need to obtain more predictive data leads to the need to build in-vitro models which best represent said microenvironment and said cell-cell interactions. This may be achieved by selecting the most suitable in-vitro context which leads to maximizing the in-vitro/in-vivo correlation, and to this end it is necessary to evaluate properties which in addition to cell death include, for example, the expression of biomarkers, in order to take into account the interaction between different cell populations and the microenvironment in which the cell is found. These properties cannot be evaluated except by means of high-content technologies.

The need is all the more felt with the entry on the market of increasingly more personalized therapies which are very expensive and the administration of which must thus be as targeted as possible. Furthermore, in many diseases, especially cancer, the rapid progression and side effects of anticancer therapies require that the therapeutic approach be the best right from the start.

US2017356911 discloses an in-vitro system which isolates PMBC (Peripheral Blood Mononuclear Cells) from a patient's blood sample and plates them in multi-well plates, preferably in 384-well plates, obtaining an experimental model which interprets the physiological context well.

Experimental works carried out over the years in multi-well plates show how, in each of the wells in which, for example, an equal volume of the same cell suspension has been seeded, different and highly complex relationships occur between the cells. Not necessarily, in each of said wells and in all the cells belonging to said wells, the optimal context is created so that the well is representative of the physiological context.

The exclusion of deviant data where the deviation is non-specific, i.e., not correlated with the analysis in progress and frequently due to the sample's non-representativeness of the physiological context, is not currently feasible with the high-content analysis platforms.

Therefore, the need to have a method which allows obtaining, on a sample of cells, an efficient, accurate and precise analysis for obtaining useful indications in clinical practice is strongly felt.

### DESCRIPTION

The present invention relates to a method for the high-content analysis of a biological sample, where said method is based on an analytical process of selection of sets or subsets of cells and/or microwells which allows defining an in-vitro model where the correlation between the measurements made by said model and the actual biological in-vivo behavior is maximized. In one embodiment, said method allows observing for example an interaction between an agent and a biological sample, eliminating interferences not associated with the action of said agent. In a further embodiment, said method allows selecting the most suitable in-vitro context so as to maximize the in-vitro/in-vivo correlation. By way of example, the method according to the present invention, providing an objective method for the removal of those data which represent effects of alteration of the cellular functionality not due to a treatment but to conditions of the in-vitro microenvironment which differ from the conditions to which the cells are subjected in-vivo, allows maximizing the correlation between the efficacy results of a treatment obtained in-vitro on a sample of patient cells and the actual clinical response to the same treatment by the same patient.

Said method is large-scale and thus allows focusing the analysis on the most suitable samples for the specific analysis to be carried out, excluding the samples which would lead to aberrant data for reasons not related to the analysis in progress but due to the sample's non-representativeness of the physiological context, while maintaining a number of data such as to ensure a statistical robustness of the result. In particular, the method according to the present invention offers the possibility of focusing the analysis on a set of microwells, each characterized by a different microenvironment due to the different relationships which are created in each of said microwells between cells and/or agents contained therein.

### Definitions

Unless otherwise defined, all the technical and scientific terms used herein have the same meaning as that commonly understood by those skilled in the art to which the present invention refers.

The term “about” of “approximately” as used herein indicates a variability within 10%, more preferably within 5%, of a given value or range.

“Microwell,” as used herein, means a receptacle which is micrometric in size (less than 1000 micrometers), including height, cross-sectional area, for example diameter where the microwell is tubular, and volume.

The term “high-content” refers to a phenotypic analysis method conducted in cells which involves the analysis of whole cells or cell components with simultaneous reading of different parameters, typically performed by acquiring images under a microscope, under phase contrast and/or fluorescence, and analyzing them.

The term “cell-cell interactions” as used herein refers to direct interactions between cell surfaces which may be stable, such as those made through cellular or transient or temporary junctions, such as those between cells of the immune system or interactions involved in the inflammation of tissues. Said interactions may also be indirect, where said cells are not in contact but are sufficiently close for the secretion of molecules, for example proteins, by a first cell to cause functional effects on a second, close cell. By way of example, following a treatment with an agent or manipulation, as in the case of CAR-Ts, T cells release cytokines which cause the death of a target which is sufficiently close.

“Treatment” refers to the therapeutic treatment of in-vitro cells or of a subject in which the goal is to improve or slow (reduce) the target disease condition or disorder, or one or more symptoms associated therewith. Said therapeutic treatment may consist of drugs or therapeutic agents.

“Response” or “responsive” refers to a cell or subject which exhibits at least one altered feature after the treatment. Similarly, “responsive to” or “responds” and similar terms refer to indications that the target disease condition, or one or more symptoms associated therewith, is prevented, improved or decreased in the in-vitro cell or in the subject. By way of example, the reduction in the number of tumor cells or a tumor mass rather than the hematological response, defined according to criteria known to those skilled in the art, are considered responses.

“Therapeutic agents” or “agent” according to the invention are a type of treatment consisting of molecules which include, without limitation, polypeptides, peptides, glycoproteins, nucleic acids, drugs of synthetic or natural origin, peptides, polyenes, macrocytes, glycosides, terpenes, terpenoids, aliphatic and aromatic compounds, and the derivatives thereof. In a preferred embodiment, the therapeutic agent is a chemical compound such as a synthetic and natural drug. In another preferred embodiment, the therapeutic agent causes the improvement and/or cure of a disease, disorder, pathology and/or symptoms associated therewith.

Suitable therapeutic agents include, without limitation, those presented in The Pharmacological Basis of Therapeutics by Goodman and Gilman or The Merck Index. The types of therapeutic agents include, without limitation, drugs which affect inflammatory responses, drugs which affect the composition of body fluids, drugs which affect the metabolism of electrolytes, chemotherapeutic agents (e.g., for hyperproliferative diseases, in particular cancer, for parasitic infections and for microbial diseases), antineoplastic agents, immunosuppressive agents, drugs which affect the blood and blood-forming organs, hormones and hormone antagonists, vitamins and nutrients, vaccines, oligonucleotides, and gene and cell therapies. It will be understood that compositions comprising combinations, e.g., mixtures or mixtures of two or more active agents, such as two drugs, are also included in the invention.

In one embodiment, the therapeutic agent may be a drug or prodrug, antibody, vaccine, or cell. The method of the invention may be used to predict whether administering a therapeutic agent to a patient will trigger a response to the therapeutic agent or to monitor a patient's response to an ongoing therapy. In a further application, said method may be used to test the efficacy of an agent on a target of potential pharmacological interest.

The nature of the therapeutic agent in no way limits the scope of the invention. In non-limiting embodiments, the method of the invention may be used to evaluate the response to small synthetic molecules, naturally occurring substances, naturally occurring biological agents or synthetic products, or any combination of two or more of the above, optionally in combination with excipients, vectors or carriers.

The term “diagnosis” refers to the identification of a molecular or pathological state, disease or condition, such as the identification of cancer, or refers to the identification of a cancer patient who may benefit from a particular therapeutic regimen.

The term “prognosis” refers to the prediction of the probability of observing or not observing a change in the state of the disease, for example a progression or regression, or the onset of certain clinical events, regardless of the specific treatment or therapeutic agent administered to a subject affected by a specific pathology.

The term “prediction” is used here to indicate the likelihood that a patient will respond favorably or unfavorably to a particular therapeutic agent. In one embodiment, the prediction relates to if and/or the likelihood of a patient surviving or improving after treatment, e.g., treatment with a particular therapeutic agent, and for a certain period of time without the progression of the disease.

The general methods and techniques described herein may be performed according to conventional methods well known in the art and as described in various general and more specific references which are cited and discussed throughout these specifications, unless otherwise indicated. See, for example, Sambrook et al., Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992); Harlow and Lane Antibodies: A Laboratory Manual, Cold Spring Harbor Laboratory Press, Cold Spring Harbor, N.Y. (1990).

A “dye” or “marker” means a molecule, compound or substance which may provide an optically detectable signal, such as a colorimetric, luminescent, bioluminescent, chemiluminescent, phosphorescent, or fluorescent signal. In a preferred embodiment of the invention, the dye is a fluorescent dye. Non-limiting examples of dyes include CF dyes (Biotium, Inc.), Alexa Fluor dyes (Invitrogen), DyLight dyes (Thermo Fisher), Cy dyes (GE Healthscience), IRDyes (Li-Cor Biosciences, Inc.) and HiLyte dyes (Anaspec, Inc.). In some embodiments, the excitation and/or emission wavelengths of the dye are between 350 nm and 900 nm, or between 400 nm and 700 nm or between 450-650 nm. In one embodiment, a marker is an antibody used to characterize the immunophenotype, a marker of viability, apoptosis, an antibody showing protein phosphorylation and pathway activation.

The term “time-lapse imaging” herein means the acquisition of multiple images of the same field carried out at successive times.

### DESCRIPTION OF THE DRAWINGS

FIG. 1: graph showing the cell mortality data (output parameter=cell death, property=% cell death) obtained by subjecting a plurality of microwells to a cell viability assay, then selected based on the cumulative derived properties “cell density in the microwell” and the relationship “average distance of each cell from the cells belonging to the same microwell.”

FIG. 2: graph showing a dose/response measurement of FLAI-5 therapy, where a plurality of microwells were subjected to a cell viability assay and the output parameter, i.e., cell death, was extrapolated from the “cell death” property measured in the areas of interest, calculating the percentage of areas of interest which have cell death marker intensity above a certain threshold and which belong to the selected set based on the cumulative derived property “number of cells per well,” i.e., said output parameter was extrapolated from the count of the fraction of the areas of interest with cell death marker intensity above a certain threshold and which belong to the set of microwells selected on the basis of the cumulative derived property “number of cells per well,” where said areas of interest are a multiplicity which encompasses all the areas of interest included in microwells containing the same number of cells per well.

FIG. 3: theoretical graph (A) and comparison between theoretical and experimental value (B) related to the co-localization obtained by sequentially seeding two homogeneous cell populations.

FIG. 4: co-localization frequency observed as the number c of cells per well varies as R1 varies

FIG. 5: co-localization frequency observed as the number of cells per well varies, with the variation of R1, for two values of R2.

FIG. 6: illustrative diagram of the steps included in the image acquisition and processing process: identification of the areas corresponding to the microwells (A); detection of a plurality of areas of interest (B); measurement of properties (columns B-G in table C) of said areas of interest (column A in table C); obtaining output parameter (column F in table D) from a set of areas of interest selected based on two of said measured properties (columns C, G in table D).

FIG. 7: block diagram of the method according to the present invention (A) and of two embodiments thereof (B, C) (μw=microwell).

FIG. 8: table showing the properties measured in step c) of the method according to the present invention.

FIG. 9: effect of an anti-CD38 agent on cell death (“−” indicates untreated microwells; “+” indicates treated microwells).

FIG. 10: diagrammatic representation of the system according to the present invention.

FIG. 11: (A) Analysis based on ICNP with selection of four subsets of microwells, where each subset satisfies one of the four inclusion criteria (patterns) described herein below. Pattern 1: subset of microwells selected to comprise at least one area of interest which satisfies the direct property “NK cell immunophenotype” and at least one area of interest which satisfies the direct property “plasma cell immunophenotype” (E/T co-localization); pattern 2: subset of microwells selected to comprise at least one area of interest which satisfies the direct property “plasma cell immunophenotype” and no area of interest which satisfies the direct property “NK cell immunophenotype”; pattern 3: subset of microwells selected to comprise at least one area of interest which satisfies the direct property “NK cell immunophenotype” and no area of interest which satisfies the direct property “plasma cell immunophenotype”; pattern 4: subset of microwells selected to not comprise any area of interest which satisfies the direct property “plasma cell immunophenotype” and any area of interest which satisfies the direct property “NK cell immunophenotype”; (B) Measurement of the distance d between an NK cell and a plasma cell, diagrammatically represented and in an original image. (C) Plasma cell mortality assessed for each of the 20 patterns identified based on the number of E (NK cells) and T (plasma cells) numbers in the same microwell. The % of wells is shown, indicating the pattern in parentheses. (D) Example of time lapse images analyzed at the single cell level. (E) Measurement of the death of target cells (plasma cells) located inside a microwell at a distance between zero (contact) and (μm) from an NK cell. The method allows estimating the fraction of active NK cells by comparing the frequency of death events where the NK cell is in contact with a target cell, with the spontaneous cell death of the target cells measured in a control represented by microwells in which E (NK cells) is null (no NK). (F) Viability of target cells expressed in % measured in the experiment in time lapse at 1, 3, 4, 5 and 6 h. The tables show the results obtained in the selected patterns, in relation to the number of NK cells and the number of plasma cells present in the microwell. A clear correlation emerges from the data, where plasma cell death increases as NK cells increase, i.e., cell mortality is higher in the lower right boxes of the graphs. The effect is already clear at an early stage (3 h). (G) (comparative) Results obtained with standard Cr51 release assay.

### DETAILED DESCRIPTION OF THE INVENTION

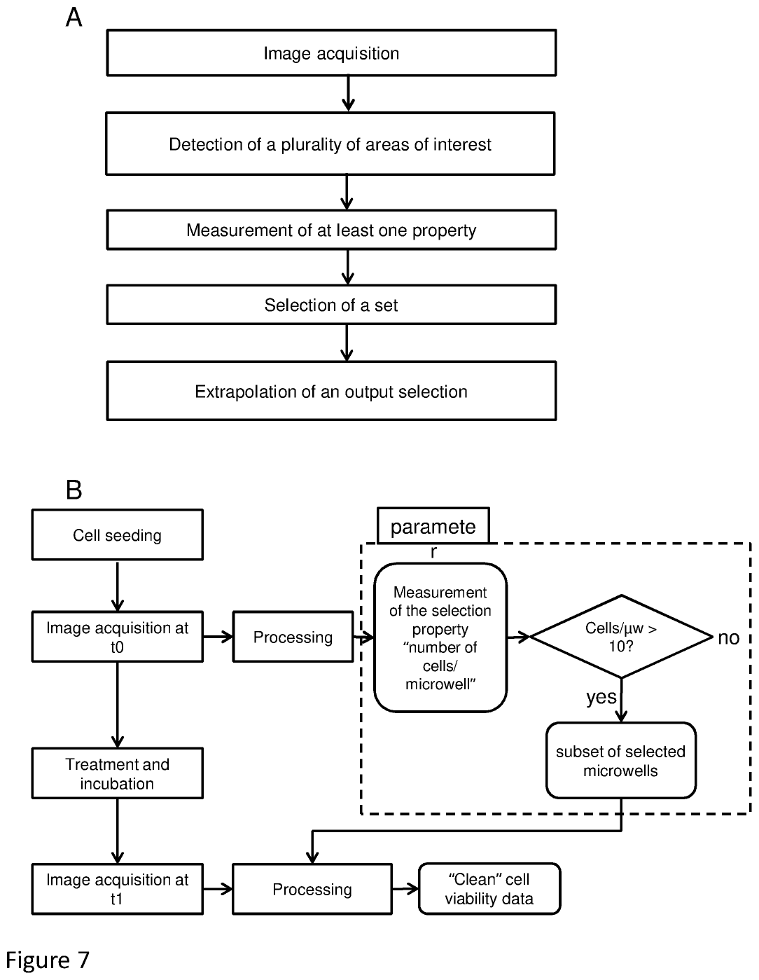

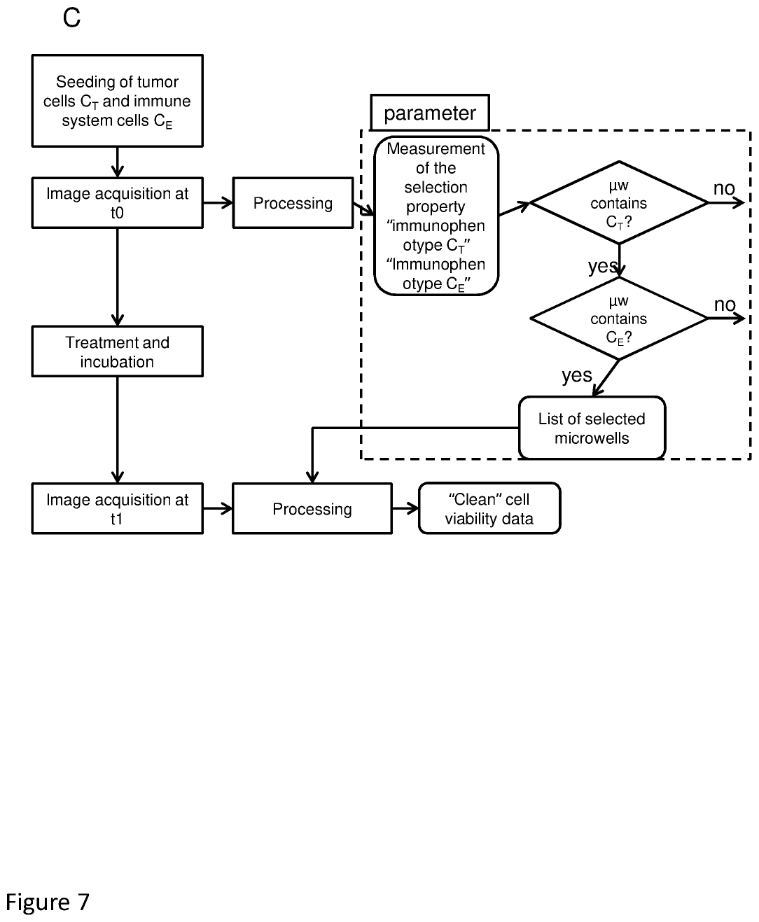

The present invention relates to, with reference to the block diagram in FIG. 7A, a method for subjecting a plurality of microwells containing cells to a high-content assay, said method comprising:
a) Acquiring at least one image of said plurality of microwells;
b) In said image, detecting a plurality of areas of interest, each area of interest corresponding to a single cell;
c) Measuring at least one property, direct or derived, of said areas of interest;
d) Selecting a set of areas of interest based on one or more of said properties, where said one or more properties are defined as selection properties;
e) Extrapolating an output parameter from a property measured in the set of areas of interest selected, where said property is defined as output property, said output property being distinct from said selection properties.

In a preferred form, in said step c), at least one derived property is measured and, optionally, at least one direct property of said areas of interest, where said one or more properties is a selection property.

In a preferred form, in said step d) a subset of said plurality of microwells is selected, where said microwells belonging to said subset contain areas of interest selected based on said at least one selection property and in said step e) an output parameter is extrapolated from a property measured in the set of areas of interest selected, where said property is defined as output property, said output property being distinct from said selection properties, where said output parameter is the processing of an output property measured in said set of areas of interest.

In a preferred form, said microwells are embedded in a plate comprising at least 15,000, or at least 18,000, preferably 19,200 microwells.

Said selection is made by imposing inclusion criteria, where said inclusion criteria comprise:
identifying, from said direct or derived properties measured, one or more selection properties;
imposing, for each of said selection properties, the threshold value, or the range of values, within which said selection property must fall.

The term “pattern” herein defines the inclusion criterion to be adopted for the selection of said set of areas of interest for a specific output parameter.

In a preferred embodiment, where a subset of said plurality of microwells is selected, said microwells belonging to the subset were selected because they contain areas of interest in which said at least one selection property satisfies said inclusion criterion.

In the present description and in the claims, the expression “property, direct or derived, of said areas of interest” has the meaning indicated below.

A direct property is a property associated with a single area of interest, i.e., a property which can be measured by assessing the single area of interest (for example, immunophenotype, cell viability, cell morphology, signaling activity).

A derived property is a property associated with a multiplicity of areas of interest, i.e., a property which, in order to be measured, requires the assessment of two or more areas of interest included in the same microwell, for example:
A property of relationship between two or more areas of interest included in the same microwell (for example, cell-cell distance); or
A property of coexistence of areas of interest, for example one or more types of immunophenotype; or
A cumulative property of all the areas of interest included in the same microwell (for example, number of cells in the microwell to which an area of interest belongs for which said cumulative derived property is calculated, average distance between the cells contained in the microwell).

Said direct properties are directly obtained from the image analysis. Said derived properties are obtained by processing direct properties. In one embodiment, where the inclusion criterion is the immunophenotype, said selection property is a direct property, the immunophenotype, and a set of areas of interest which satisfy the inclusion criterion is selected.

Still maintaining the immunophenotype as inclusion criterion, in an embodiment, where at least a subset of said plurality of microwells is selected, said selection property is a derived property, where said derived property is a coexistence property, i.e., a property generated by evaluating the direct immunophenotype property in each area of interest included in a single microwell, and by processing the direct properties of each of the areas of interest contained in a microwell, by extrapolating the derived property which is the peculiar immunophenotypic pattern of the microwell which, based on this pattern, will be attributed to a subset of said plurality of microwells.

As a further example, the cell-cell distance is a derived property, obtained by processing the direct “position” properties associated with two areas of interest included in the same microwell. From a multiplicity of said “cell-cell distance” derived properties, a further derived property is obtained which is a further relationship property, i.e., the average distance between the cells contained in a given microwell surrounding a selected cell, from said selected cell. A further derived property is also derived which is a cumulative property of all the areas of interest included in the same microwell to which a given area of interest belongs, i.e., the average distance between the cells contained in a given microwell. It is also possible to determine further properties derived from the combination of direct properties with relationship properties. For example, the derived property “distance of an immune cell from a tumor cell” requires combining the direct property “immunophenotype” with the relationship property “cell-cell distance.”

Is should be noted that the derived properties are also properties of the areas of interest. Some cells belonging to the same microwell have the same relationship-derived property value. All the cells belonging to the same microwell have the same cumulative derived property value. For example, two cells contained within the same microwell have the same derived property “cell-cell distance” value when this is calculated between said two cells. Furthermore, the relationship property “average distance between cells contained in a given microwell surrounding a cell selected by said selected cell” takes on a different value for each selected cell, since, for each selected cell, the distance from the other cells in the same microwell surrounding it will be different. Again, all the cells belonging to the same microwell have the same cumulative derived property “number of cells per microwell” value, meaning that this property of the context in which each cell is placed (the microwell) is made its own by each cell, i.e., by each area of interest, belonging to the microwell itself. In this case, or when a cumulative property is discussed, equal for each area of interest embedded in the same microwell, this property may be considered a property of the microwell, meaning that this property applies to all areas of interest embedded within said microwell.

Said set of areas of interest comprises:
a subset of two or more areas of interest not embedded in the same well; and/or
a subset of two or more areas of interest included in the same microwell; and/or
a subset of all the areas of interest included in the same microwell.

In a preferred form, said set of areas of interest consists of a subset of all areas of interest included in the same microwell, i.e., said set of areas of interest corresponds to a subset of microwells.

In one embodiment, at least one of said selection properties is a coexistence property.

In one embodiment, at least one of said selection properties is a cumulative property of all the areas of interest included in the same microwell.

In the present description and in the claims, the expression “output parameter from a property measured in the selected set of areas of interest” will indicate the result of any statistical processing of the output property measured in each area of interest belonging to said selected set. “Statistical processing” means, for example, the mean value, the median, the mean square value, etc.

In the embodiment where one or more of said selection properties is a cumulative property of all the areas of interest included in the same microwell, said set of areas of interest corresponds to a subset of said plurality of microwells and said output parameter is the processing of an output property measured in said subset of said plurality of microwells.

It is understood that said selection, in one embodiment, comprises a selection of a first set of areas of interest based on a first selection property. This is followed by a selection, within said first set of areas of interest, of a subset of areas of interest based on a second selection property. Said first and second selection properties are independently direct or derived properties. In a preferred form, said set of areas of interest and/or said subset of areas of interest corresponds to a subset of the plurality of microwells.

In a further embodiment, said process comprises a first selection, a second selection and a third or further selections.

Said at least one image is acquired with an image acquisition device configured to acquire at least one image of said plurality of microwells.

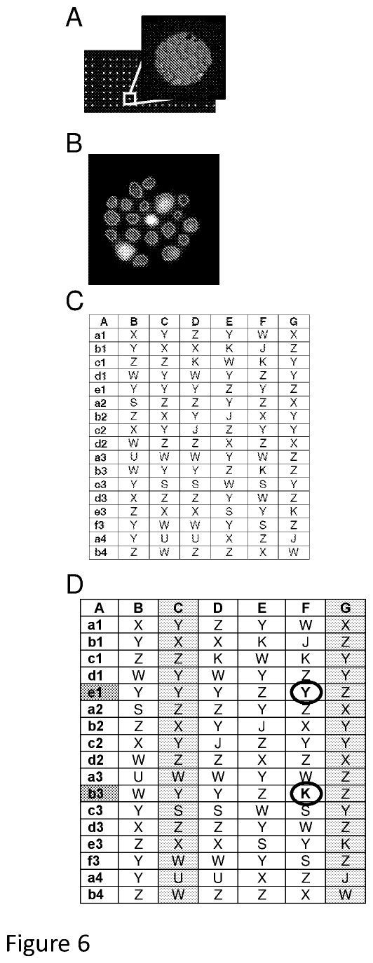

In one embodiment, the image analysis and processing process comprises the following steps, with reference, where appropriate and purely for explanatory purposes and not in any way limiting the scope of the invention, to FIG. 6:
In an image containing a plurality of microwells, the zones corresponding to the microwells are identified (FIG. 6, panel A);
Within the zones corresponding to the microwells, a plurality of areas of interest are detected, each area of interest corresponding to one of said cells contained in said plurality of microwells (FIG. 6, panel B);
At least one property of said areas of interest is measured (FIG. 6, panel C; column A: areas of interest; columns B, C, D: direct properties; columns E, F, G: derived properties);
A set of areas of interest is selected based on one or more of said properties (FIG. 6, panel D; the columns of the selection properties are highlighted in gray, the set of areas of interest selected is highlighted in dark gray);
An output parameter is extrapolated from properties measured in said set (FIG. 6, panel D; the output properties are surrounded).

With reference to FIG. 6, panel D, the inclusion criterion is: property C=Y and property G=Z. The output parameter is extrapolated from property F. Being enclosed in a circle, the properties F related to the set of selected areas of interest are thus highlighted. The result of a statistical processing of said output property F measured in said set of areas of interest is the output parameter provided by the method according to the present invention, representative of the output property F in analysis of the sample under examination.

It should be noted here that, for simplicity, the diagram in FIGS. 6C and 6D comprises a limited number of areas of interest, where in the implementation of said method the areas of interest are advantageously very numerous. By way of example, where said plurality of microwells corresponds to a plate of 19,200 microwells, assuming an average of about 20 cells/microwell, 384,000 areas of interest are available.

The acquired images are analyzed by a computer, with the aid of suitable software products for image processing. Such software products are for example ImageJ, BiolmageXD (Kankaanpaa P et al. Nature Methods. 2012), Icy (De Chaumont F et al. Nature Methods. 2012), Fiji (Schindelin J et al. Nature Methods. 2012), Vaa3D (Peng H et al. Nat Biotechnol. 2010), CellProfiler (Carpenter AE et al. Genome Biol. 2006), 3D Slicer, Image Slicer, Reconstruct (Fiala JC. J Microsc. 2005), FluoRender, ImageSurfer, OsiriX (Rosset A et al. J Digit Imaging. 2004), IMOD (Kremer JR et al. J Struct Biol [Internet]. 1996) among others (Eliceiri KW et al. Nature Methods. 2012).

Those skilled in the art can easily understand that the above software products are exemplary only and that the method may be carried out using approaches not explicitly mentioned here, providing the same type of result.

In a preferred form, said plurality of microwells is embedded in a microfluidic device where each microwell is in fluid communication with one or a plurality of microchannels for the delivery of fluids and/or particles and/or molecules to the wells.

In one embodiment, the microwells are inverted open microwells, i.e., they are microwells with both an upper end and a lower end open, preferably said ends being open on one or more microchannels in which a fluid is present, a fluid comprising cells or particles or molecules, or air or other gases.

The microwell has a vertical axis, such as a central axis, which extends between the top and bottom of the microwell. In one embodiment, said microwell is open at the upper end on a microchannel, called upper microchannel, which comprises a fluid and, at the lower end, on a microchannel in which air or other gases is present. In this embodiment, the fluid inserted into the microchannel fills the microwell through capillary action, while the surface tension holds the fluid inside the open microwell, forming a meniscus at the air/fluid interface.

In one embodiment, said microwells are sized so as to have a height equal to or greater than the diameter thereof.

In an even more preferred form, said microwells are microwells of the type described in the application WO2012072822.

Said cells are seeded in said microwells according to methods known to those skilled in the art, and are a homogeneous cell population, i.e., they have the same immunophenotype, or heterogeneous, i.e., with a different immunophenotype.

In a preferred form, said cells are seeded according to the method described in WO2017216739.

Said cells are seeded in a single step, or in sequence. By way of example, using inverted open microwells it is possible to load populations which are different from each other in sequence and each of which contains cells which are homogeneous to each other, creating heterogeneous populations in the volumes in which the cells are deposited.

By way of example, using microwells with a diameter of 70 μm, up to 20, up to 30 or up to 50 cells/microwell are seeded.

In one embodiment, a heterogeneous cell population is seeded on a subset of microwells in a single step. In a further embodiment, several seeding processes are carried out in sequence. By way of example, a first seeding of a population 1 which is at a concentration c1 and a second seeding with a population 2 at a concentration c2 are performed. Where said concentrations c1 and c2 are equal, seeding equal volumes will result in a heterogeneous population in the set of microwells belonging to the subset where on average the number of type 1 cells is equal to the number of type 2 cells. The cells will instead be present inside the microwells according to a distribution, typically a Poisson distribution, which sees a variable number of type 1 and type 2 cells. Some wells will contain only type 1 or 2 cells, others will contain both types, and still others may be empty. By seeding a double volume of the type 1 population, a heterogeneous population will be obtained in the set of microwells belonging to the subset where on average the number of type 1 cells is double compared to the number of type 2 cells. The distribution of type 1 cells in the microwells, compared to the previous case, will see a doubled average value.

In one embodiment, said method is carried out on the same plurality of microwells at successive and repeated times. That is, in this embodiment, images are acquired, a multiplicity of areas of interest detected and the at least one property measured at time t₀ and, subsequently, at time t₁, t₂, . . . tₙ. In this embodiment, the assay is defined as dynamic, i.e., multiple images of the same field are acquired at successive times (time-lapse imaging) and the measurement of said at least one property, at time t₀ and, subsequently, at time t₁, t₂, . . . tₙ, returns an analysis which reflects variations over time.

Said property at t₀ is to be understood as distinct from said property at t₁. I.e., assuming to measure the property C (P_{c}), P_{ct0} and P_{ct1} are clearly to be intended distinctly.

As a result, within the execution of the same assay, said output parameter may be derived from the output property P_{ct1} in the set of areas of interest selected, where a selection property was P_{ct0}.

In a further embodiment, a derived property is a variation (e.g., the difference, or ratio) between the property measured at t₀ and the property measured at t₁, or vice versa.

In one embodiment, said cells, while they are kept in said plurality of microwells, are exposed to one or more agents which promote or inhibit an objective effect of the analysis, i.e., which impact the output parameter. The dynamic method according to the present invention allows determining the effect of said agent over time.

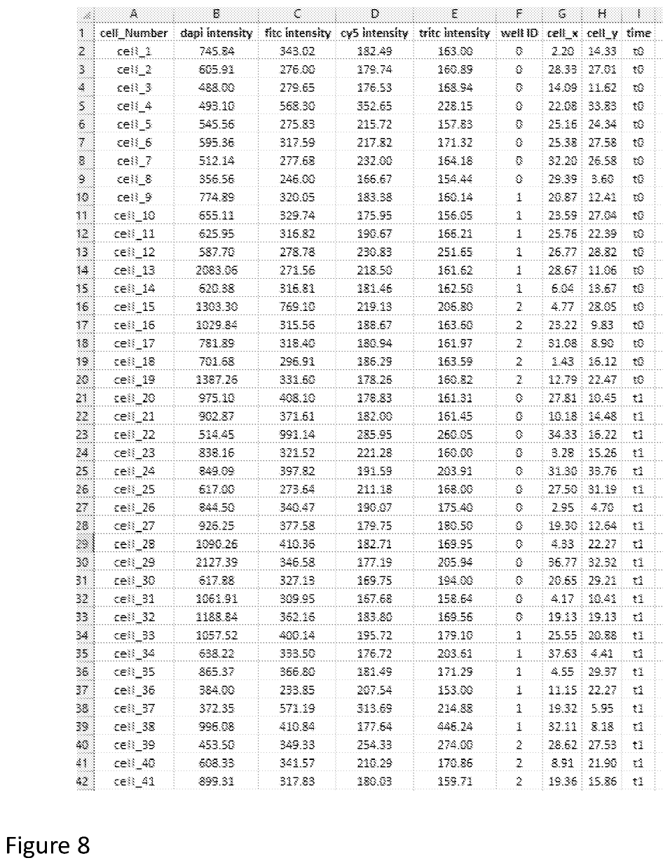

By way of example, and with reference to the table in FIG. 8, for each area of interest corresponding to a cell (column A), the direct properties “DAPI signal intensity,” “FITC signal intensity,” “Cy5 signal intensity,” “TRITC signal intensity,” “cell position on the X axis and Y axis” (columns B-E, G, H) at t₀ (lines 2 to 20) and the same properties at t₁ (lines 21 to 42) were measured. The microwell to which each cell belongs is also reported (column F). Combining said direct properties referred to in columns B-E, G, H with the information related to the microwell to which each cell belongs, it is possible to calculate derived properties, for example for each cell it is possible to determine the average distance from other cells contained in the same microwell from said cell.

Properties

In the following paragraphs, some categories of properties are listed, providing some technical-experimental details which allow measuring them. Downstream of each of said procedures, it is understood that an image acquisition and processing step by means of the above computational approaches is included, which is capable of returning the information related to the specific property which is typically information of a numerical type.

The following list is illustrative and must in no way be construed as limiting the technical-experimental approaches described for each property. Given a property, those skilled in the art know the most suitable experimental approach to give evidence thereof. Furthermore, this list must not be construed as limiting the possible properties. Those skilled in the art know how to extend the list with further direct or derived properties to be effectively measured in the method according to the present invention.

It is understood that said properties may independently constitute selection properties or output properties.

Immunophenotype (Direct Property)

It may be determined and/or verified using methods known in the art. For example, using detectable markers/dyes. Such markers/dyes may be specific for one or more subpopulations embedded in the microwells. Where specific markers/dyes are used, these may be selected to highlight cell populations which play a role in various diseases. For example, because they are responsible for a tumor, for example a blood cancer, or because they are responsible for an inflammatory and/or immune response.

The staining may comprise the use of multiple detectable markers, for example, cells may be stained with a primary antibody which binds to a specific target antigen and a secondary antibody which binds the primary antibody or a molecule coupled to the primary antibody may be coupled to a detectable marker. The use of indirect coupling may improve the signal-to-noise ratio, for example by reducing the background binding and/or by providing signal amplification.

The staining may also comprise a primary or secondary antibody directly or indirectly coupled to a fluorescent marker. By way of non-exhaustive example, the fluorescent marker may be selected from the group consisting of: Alexa Fluor 350, Alexa Fluor 405, Alexa Fluor 430, Alexa Fluor 488, Alexa Fluor 514, Alexa Fluor 532, Alexa Fluor 546, Alexa Fluor 555, Alexa Fluor 568, Alexa Fluor 568 594, Alexa Fluor 610, Alexa Fluor 633, Alexa Fluor 635, Alexa Fluor 647, Alexa Fluor 660, Alexa Fluor 680, Alexa Fluor 700, Alexa Fluor 750 and Alexa Fluor 790, fluorescein isothiocyanate (FITC), Texas Red, SYBR Green, Fluidi DyLight, green fluorescent protein (GFP), TRIT (tetramethyl rhodamine isothiol), NBD (7-nitrobenz-2-oxa-1,3-diazole), Texas red dye, phthalic acid, terephthalic acid, isophthalic acid, fast cresyl violet, cresyl blue violet, brilliant cresyl blue, para-aminobenzoic acid, erythrosine, biotin, digoxygenin, 5-carboxy-4′, 5′-dichloro-2′, 7′-dimethoxy fluorescein, phthalocyanines, azomethines, cyanines (e.g., Cy3, Cy3.5, Cy5), xanthines, succinyl fluorescein, N, N-diethyl-4-(5′-azobenzotriazolyl)-phenylamine, aminoacridine, brilliant Violet 421, phycoerythrin (PE).

Number of cells/microwell (derived property, cumulative of all areas of interest included in the same microwell)

Before seeding or when seeded in the microwells, the cells are stained with a dye such as the fluorescent cell localization marker 7-amino-4-chloromethylcoumarin.

Cell-to-cell distance (direct property associated with derived, relationship property)

Before or after seeding, the cells are stained, possibly with a staining which differentiates them according to the immunophenotype, and through the above image processing approaches, a direct property is obtained for each cell which is the position of said cell in space. Combining said direct property “position” associated with an area of interest with said direct property “position” associated with a different area of interest, the derived property of the desired relationship is obtained, i.e., the cell-cell distance.

Cell Viability (Direct Property)

Known markers/dyes are used which specifically recognize the cells which are at a particular stage of the cell cycle. These include, by way of example, selective markers for cells with non-intact membranes or selective markers for cells in an advanced stage of cell death or early apoptosis. For example, it is possible to use antibodies against cytochrome C, causing DNA turnover, or dyes which cause cell viability/death such as propidium iodide (PI) and calcein, or dyes which cause cell proliferation, or apoptosis markers such as Annexin V, or dyes which cause apoptosis by means of the measurement of the signaling and release activity of certain proteins and enzymes, such as caspases. Preferably, said markers/dyes are added to the cells in the microwell.

### Signaling Activity (Direct Property)

Preferably already in the microwell, the cells are labeled with markers such as to highlight cellular signaling, such as antibodies capable of highlighting the phosphorylation of proteins or the release of calcium ions in the cytoplasm. In one embodiment, the “signal intensity” property is determined by time-lapse imaging at t₀ and “signal strength” at t₁, t₂, . . . tₙ associated with the marker used and cells are selected having the variation of said “signal intensity” property over time beyond a certain threshold value.

### Cell Morphology (Direct Property)

The image of the cells, possibly stained according to one of the methods described and known in the state of the art, is acquired and processed through the computational approaches mentioned above, returning the information about the cell morphology.

In a preferred embodiment, said selection is made based on at least 2 selection properties, or at least 3, or at least 4, or at least 5 selection properties.

One or more of said selection properties lead to selecting a set of areas of interest which, in a preferred form, correspond to a subset of microwells from which the output parameter will be derived.

A well-defined pattern allows optimizing the assay result.

Those skilled in the art know how to establish the pattern best suited to the output parameter of interest.

By way of example, where the assay is conducted to measure cell death in a sample, those skilled in the art, knowing that cell viability is negatively affected by being in an isolated microenvironment and not with other neighboring cells, establishes that at least one of said selection properties is the number of cells/microwell, imposing a minimum threshold value X for this property. Therefore, the pattern will be: microwell cell number >X. The result will derive from extrapolating, from the set of microwells which satisfy the established pattern, the output parameter.

In one embodiment, said patterns are advantageously established using the method according to the present invention, so as to make them optimal for the specific sample on which the assay is conducted. As an example, in an assay, control subset(s) are used in which an output parameter is optimized and subsequently these control values are also used for the classification of the subgroups exposed to treatment. For example, in a plurality of microwells containing cells not exposed to any agent, the minimum number of cells for each microwell is determined, which allows obtaining a minimum mortality at 24 h (number of cells at t₀). This threshold value of the selection property “number of cells” at t₀ is used to select the set of areas of interest exposed to a drug, and therefore the subset of microwells exposed to a drug, in which the output property will be read and then the output parameter which is the mortality at 24 h (number of cells at t₂₄ₕ) will be extrapolated. I.e., the output parameter “number of cells” at t₂₄ₕ will be the result of the statistical processing, in the specific case the average value, of the output property “number of cells” at t₂₄ₕ measured in each of the microwells belonging to the subset of microwells exposed to the selected drug because they satisfied the pattern, i.e., showed, at t₀, a number of cells above the threshold value as defined above. Thereby the pattern is optimized based on the biological features of a specific sample.

In a further aspect, with reference to FIG. 10, a system (1) for subjecting a plurality of microwells containing cells to a high-content assay is claimed, said system comprising:
an image acquisition device (2) configured to acquire at least one image of said plurality of microwells (3); and
a data processing unit (4) configured to:
   In said image, detecting a plurality of areas of interest, each area of interest corresponding to a single cell;
   Measuring at least one property, direct or derived, of said areas of interest;
   Select a set of areas of interest based on one or more of said properties, where said one or more properties are defined as selection properties;
Extrapolating an output parameter from a property measured in the set of areas of interest selected, where said property is defined as output property, said output property being distinct from said selection properties.

In a preferred form, said processing unit is configured to measure at least one derived property, and, optionally, at least one direct property, of said areas of interest, where said one or more properties is a selection property;

In a preferred form, said processing unit is configured to select a subset of said plurality of microwells, where said microwells belonging to the subset contain areas of interest selected based on said at least one selection property.

In a preferred form, said processing unit is configured to extrapolate an output parameter from a property measured in the set of areas of interest selected, where said property is defined as output property, said output property being distinct from said selection properties, where said output parameter is the processing of an output property measured in said set of areas of interest.

In a further aspect, a computer program is claimed for subjecting a plurality of microwells containing cells to a high-content assay, said computer program comprising instructions which, when the program is executed by a data processing unit, cause the processing unit to perform the following steps:
In at least one image of said plurality of microwells, detecting a plurality of areas of interest, each area of interest corresponding to a single cell;
Measuring at least one property of said areas of interest;
Selecting a set of areas of interest based on one or more of said properties, where said one or more properties are defined as selection properties;
Extrapolating an output parameter from a property measured in the set of areas of interest selected, where said property is defined as output property, said output property being distinct from said selection properties.

In a preferred form, said computer program comprises instructions which, when the program is executed by a data processing unit, cause the processing unit to perform the following steps:
Measuring at least one derived property, and, optionally, at least one direct property of said areas of interest, where said one or more properties is a selection property;
Selecting a subset of said plurality of microwells, where said microwells belonging to the subset contain areas of interest selected based on said at least one selection property;
Extrapolating an output parameter from a property measured in the set of areas of interest selected, where said property is defined as output property, said output property being distinct from said selection properties where said output parameter is the processing of an output property measured in said set of areas of interest.

### EMBODIMENTS

In an embodiment, with reference to the block diagram in FIG. 7B, said selection is carried out based on the selection property “number of cells contained in a microwell” t₀ and said output parameter is extrapolated from the “cell viability” output property at t₁ measured in the set of areas of interest which corresponds to the subset of microwells in which said selection property is greater than a threshold value at t₀. In this embodiment, said output property is measured at a time t₁ later than the time t₀ for measuring said selection property, after having exposed the cells to an agent which influences cell viability. Assuming that the optimal condition for the growth of said cells requires having at least 10 cells in a microwell, since having less than 10 cells leads to non-negligible cell death in the microwell, the subset of microwells to which those microwells comprising more than 10 cells belong will be selected. Said output parameter is extrapolated from said subset. The cell viability datum thus obtained is a “clean” datum, i.e., not affected by the readings in those wells containing less than 10 cells which are to be considered outlier readings, since they carry therewith a high cell death independent from the agent to which the cells were exposed but linked to the experimental condition thereof.

In a further embodiment, with reference to the block diagram in FIG. 7C, said selection is made in three steps.

In the first step, a first set of areas of interest is selected based on a direct property “immunophenotype C_{T}” of the areas of interest at t₀. Said first set of areas of interest corresponds to the subset of the plurality of microwells comprising those microwells in which there is an area of interest which satisfies said selection property, or in which there is at least one cell with immunophenotype C_{T} at t₀.

In the second step, in said subset of the plurality of microwells, a second subset is selected based on a direct property “Immunophenotype C_{E}” of the areas of interest at t₀, said second subset will thus comprise those microwells which have at least one cell with immunophenotype C_{T} and at least one cell with immunophenotype C_{E} at t₀.

In the third step, in said second subset a third subset is selected based on the direct property “immunophenotype C_{T}” of the areas of interest at t₀, said third subset will thus comprise cells with immunophenotype C_{T} which are found in microwells which also comprise cells with immunophenotype C_{E}.

The output parameter is then extrapolated from the property “cell viability” at t₁ measured in said third subset of selected areas of interest. That is, said output parameter is extrapolated in relation exclusively to cells with immunophenotype C_{T} contained in microwells which see the simultaneous presence at t₀ of cells with immunophenotype C_{E}. In this embodiment, said output parameter is provided at a time t₁ later than the time t₀ for measuring said selection properties, after having exposed the cells to an agent which influences the viability of the cells C_{T}, the activity of said agent being mediated by the cells C_{E}.

This embodiment is particularly advantageous in carrying out an assay which measures the efficacy of an agent which is an immunotherapy, i.e., which acts on a target by promoting the activity of the immune system cells towards said target. The method according to the present invention advantageously allows excluding from the result the microwells which, not comprising cells of the immune system, would inevitably return a negative datum, i.e., a lack of response to the immunotherapeutic agent, where said lack of response would not be linked to an ineffectiveness of the compound under analysis but to the sample which is not suitable for the analysis itself, i.e., a datum which if it were positive would be linked to a mechanism of direct action of the drug against the target and not mediated by the cells of the immune system.

In a further embodiment, said output parameter is extrapolated in relation exclusively to cells with immunophenotype C_{T} contained in microwells which see the simultaneous presence at t₀ of cells with immunophenotype C_{E} and the distance of which from cells with immunophenotype C_{T} is less than a predetermined threshold value. This embodiment is particularly advantageous when the agent for which the efficacy is to be evaluated involves a contact or a high proximity between cells with immunophenotype C_{T} and C_{E} so that the agent may exercise the action thereof.

Where each of the analyzed cells has a potential agonist or antagonist role with respect to the effect of the assay, advantageously said selection property is a relationship property, for example cell-cell distance, signaling activity. By way of example, where cells of the immune system have a potential antagonistic effect with respect to the viability of tumor cells, the assay is effectively conducted on a set of areas of interest identified according to the method of the present invention after a selection based on derived selection properties, of coexistence, “tumor immunophenotype” and “immune system cell immunophenotype” so as to comprise cells of the immune system and tumor cells, and a derived selection property “cell-cell distance”, with a pattern thus imposing that tumor cells and immune system cells are at a distance such as to allow an interaction therebetween. In one embodiment, the pattern imposes that the aforementioned distance be such as to produce contact between an immune cell, for example a natural killer cell (NK), and a target cell, for example a tumor cell. In another embodiment, the pattern imposes that the aforesaid distance is equal to or greater than the distance which allows contact between the immune cell and a target cell since the functional effect is generated by secretion products, for example cytokines produced by T lymphocytes, which exert an effect on the target cell even in the absence of contact, as long as the distance between the two types of cells is sufficient to ensure that the concentration of the products secreted by the immune cell is significant to produce the desired effect.

In one embodiment, the immune cells are modified before the analysis by means of known processes, being for example CAR-T cells, NK cells destined for an autologous transplant, and the analysis described herein aims to verify the effective ability of the modified cells to produce a desired effect on target cells.

Again, the cell-to-cell distance, assessed at t₀ and at t₁, before and after the addition of one or more agents in said plurality of microwells, allows verifying the changes of the cell-cell interactions due to the one or more agents.

For example, in a further embodiment the plurality of microwells is first divided into homogeneous subgroups, for example 2, or 3, or 4, or 16, or 32, or 64, or 96, or 128, or 384 subgroups, and on each of said subgroups a different treatment is tested, where each treatment is defined by a specific agent at a specific dosage. The microwells belonging to each of the subgroups are selected for a direct selection property “immunophenotype” at t₁ and the output parameter is extrapolated from the property “cell viability” measured in the set of areas of interest selected. The method according to the present invention, being capable of being implemented on plates containing 19,200 microwells, and allowing the automated analysis, allows a multiplicity of different conditions to be tested in each experimental plate, for example up to 16, or up to 32 different experimental conditions, where hundreds or thousands of microwells are dedicated to each experimental condition. In one embodiment, the plates contain 1,200 wells for each condition and the plurality of microwells are exposed to 2 or 3 or 4 or 16 or 32 or 64 or 96 or 128 or 384 different conditions. The data obtained in each microwell belonging to the same subset are processed with a statistical analysis so as to return the result of the analysis. By way of example, where the agents tested were tested for the ability to cause cell death in tumor cells, an output parameter is extrapolated from the property “cell viability” measured in each subset of microwells and the subset in which the greatest degree of cell death is indicative of the most suitable agent, where the most suitable agent means the agent which may be most effective in causing the in-vivo cell death of tumor cells in the patient from whom said cells were taken or, more in general, the agent which causes the desired effect on the biological sample tested, having excluded causes other than the action of the drug itself which could cause a variation of the output parameter from which the desired effect is deduced. The number of microwells for each of the experimental conditions allows maintaining a high statistical significance even if, following the selection made according to the aforementioned selection properties, the number of wells actually subjected to the analysis is significantly reduced. The availability of a large number of microwells thus represents a fundamental requirement for supporting the method discussed herein, where the actual number of wells is strictly connected to the type of analysis. In order to ensure statistical significance, the output parameter(s) must be read on a sufficient number of samples. Typically, a sufficient number of samples is at least 30, or 100 or 300.

The selection of a subset of microwells advantageously allows testing an effect in a subset of microwells, where said selection has been carried out based on a pattern, i.e., homogeneous features of the selection properties considered.

In one embodiment, the pattern is determined in a control subset not exposed to any agent, in order to ensure optimal functional features in the control sample itself. Subsequently, said pattern is also imposed on the subsets subjected to different in-vitro treatments, or treated with different therapeutic agents possibly at different dosages. Said optimal functional features are obtained, for example, through the maximization of the cell viability, the maximization of the cell proliferation rate, obtaining a cell proliferation rate similar to the expected proliferation rate in the body from which the cells under analysis were extracted, obtaining a cellular composition, i.e., the related ratio between cells having different immunophenotype, or belonging to different cell populations, similar to that observed in said organism.

In a further embodiment, where it is desired to determine as a selection parameter the signaling in response to an agent, the intensity of the signal associated with a marker is observed at subsequent times through time-lapse imaging. Once a threshold value has been defined, the subset of microwells is selected where one or more effectors have produced a functional effect in the presence or absence of a certain agent.

### Advantages

The method of the present invention is carried out in microwells and, with the data acquisition and processing method described herein, conveniently allows observing and processing all the information related to each of the cells contained in each microwell. This means having all the information of a niche, where a niche herein means the microenvironment occupied by the cell population. Advantageously, this information allows defining a pattern, and therefore the output parameter is assessed in the context in which the assay is conducted.

The method advantageously allows carrying out assays on a sample purged of data which would introduce deviations with respect to the measurement of the analysis or which would introduce additional factors in the analysis, thus increasing the variability of the result. Therefore, the method according to the present invention allows excluding from the assay those microwells and possibly those cells which, for reasons independent of the assay to be conducted, are identified as outliers. Since said selection is made thanks to a pattern which is optimal for what is defined above, said selection made on the sample is absolutely controlled and objective and maximizes the in-vitro/in-vivo correlation.

Optionally, once the microwells of interest have been selected, the method allows for a further selection at the cellular level, thus excluding cells which behave as outliers inside microwells, thus allowing further refinement of the analysis.

Assays conducted on subsets of microwells selected according to the method of the present invention, ensuring a sufficient parallelism of the analysis by performing it on a sufficiently large number of microwells, lead to results with a high level of statistical significance despite the application of selection criteria which reduce the number of data actually considered in the analysis. For example, where the assay involves the assessment of an agent which causes death in tumor cells, carrying out the assay in microwells comprising a few cells, distant from each other, would in some cases inevitably lead to the reading of an effect on cell viability, where said effect is not at all indicative of the activity of the tested agent but is related to the experimental in-vitro conditions to which the specific sample under examination is exposed and which introduce artificial effects of toxicity towards the sample which are not due to the drug. Such artificial effects, if not eliminated from the analysis, would lead to an erroneous conclusion with respect to the measurement of the actual efficacy of the drug.

Furthermore, the method according to the present invention allows measuring and processing said properties in an automated manner, processing the acquired images and processing the data obtained by a computer.

The combination of these features ensures that the number of samples tested is such as to ensure a statistically significant datum.

Therefore, the present invention provides a method which allows the use of physiologically relevant, multi-population cell samples in studies which allow defining, by way of example, the biological effects of drug-based therapies on cellular samples, based on accurate analyses at the single cell level, thus allowing the prediction, with a quick and accurate ex-vivo analysis, of the drug which will prove to be the most effective in the subject under analysis.

The following examples have the sole purpose of illustrating the invention, and do not in any way limit it, the scope of which is defined by the claims.

### Example 1: Cell Death Control

Cells of the HL-60 cell line are plated in culture medium in inverted open microwells of a microfluidic device with 19,200 microwells. At t₀ the cells are labeled with a cell death marker (propidium iodide, PI) kept in the culture medium for the entire duration of the experiment and with a fluorescent cell localization marker (7-amino-4-chloromethylcoumarin). Images are then acquired after a 24-hour incubation (t₂₄) and a range of properties are measured in the areas of interest.

The selection properties used in this example were:
cumulative derived property: number of cells contained in each microwell;
derived relationship property: average distance of each cell from the other cells belonging to the same microwell.

The extrapolated output parameter is cell mortality (expressed as % of dead cells, i.e., cells for which the intensity of the fluorescence signal emitted by the PI marker exceeds a certain threshold).

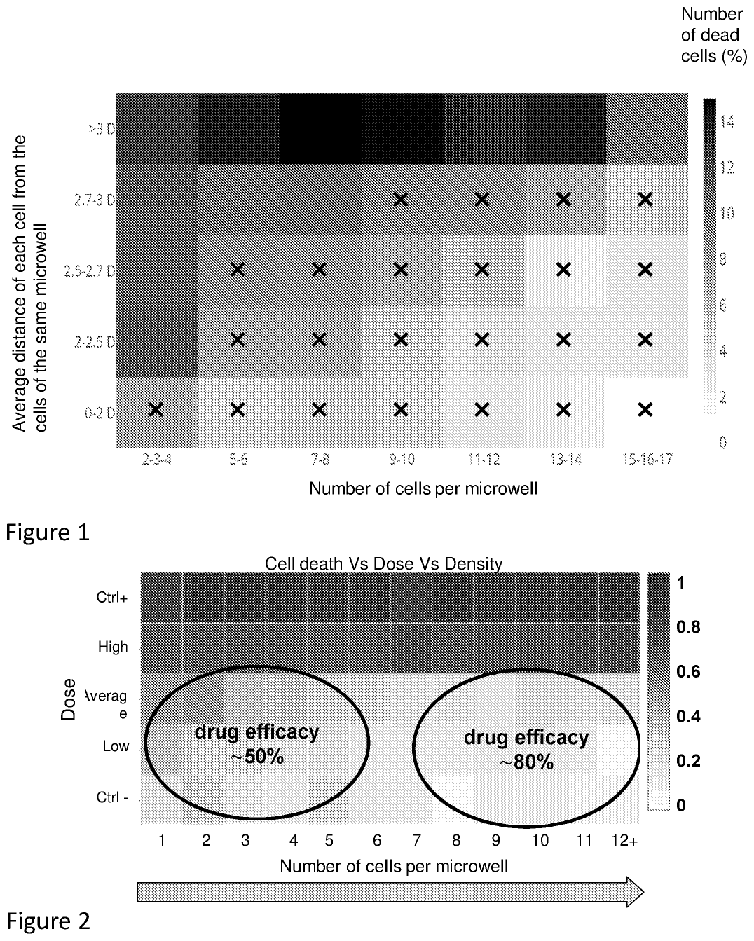

With reference to FIG. 1, classes are identified for the selection property “number of cells per well”, in particular 7 classes are determined for values equal to 2-4, 5-6, 7-8, 9-10, 11-12, 13-17, 15-17 cells/microwell, datum reported on the x-axis of the graph in FIG. 1. Within each well, a classification is then performed for the relationship-derived selection property “average distance of each cell from the cells of the same well,” obtained from the average of the distances between each cell and the cells present in the same well. The plurality of microwells is thus classified into subsets which include cells in contact, in which the average distance of the cells of the same microwell is between 0 and 2 D, where D means the average diameter of the cell under analysis, and with cells not in contact and which see cells of the same microwell gradually more and more distant, in which the average distance is between 2 and 2.5 D, between 2.5 and 2.7 D, between 2.7 and 3 D, and greater than 3D, datum reported on the y-axis of the graph in FIG. 1.

The output parameter, i.e., cell mortality, is extrapolated in each of the above subsets. Said output parameter is indicated with the gray scale in FIG. 1.

Surprisingly, cell mortality is observed to have a gradient behavior with respect to the two imposed selection properties. In fact, an increased cell death (darker color in the graph) is observed in the set of areas of interest which correspond to the subset of microwells containing fewer cells and/or in the set of areas of interest for which the average distance from the cells of the same microwell is higher. With the same number of cells contained, cell death is in fact greater for those cells which are further away from other cells.

By defining a maximum mortality which is accepted as tolerated as an artificial effect, the assay in this example allows defining, afterwards and for the purposes of subsequent analysis, the optimal pattern, establishing the threshold value for the selection property “number of cells/microwell” and the threshold value for the property “average cell-cell distance,” where said threshold values are those which allow keeping mortality within the tolerated limits.

By way of example, assuming that the tolerance limit is a maximum mortality of 10%, the subsets of microwells which meet this criterion are those highlighted with the symbol (x) in FIG. 1A. The pattern which identifies the subsets of microwells of interest is thus defined by the following relationship:

(N≥9 and P≤3D) or (N≥5 and N≤8 and P≤2.7D) or (P≥2D)

having indicated with N the property “number of cells per microwell” and with P the property “average cell-cell distance.”

As established above, the pattern is conveniently applied in the execution of a response assay to an agent which impacts cell viability, as in example 2 below. In a dose-response analysis, a reference analysis is thus normally carried out on a control, for example the sample kept in optimal conditions to ensure maximum viability and in the absence of agents, from which said pattern is determined. The analysis is also conducted in other conditions which see the administration of an agent at one or more dosages, where the analysis of the drug's efficacy is carried out on the subset of areas of interest identified based on the pattern defined by said reference analysis on a control.

### Example 2: Efficacy Analysis of a Pharmacological Agent

Cells of the HL-60 cell line are plated in inverted open microwells of a microfluidic device with 19,200 microwells in culture medium and exposed to treatment with FLAI-5: Fludarabine (FL)+Ara-C(A)+Idarubicine (I) at 3 different concentrations (low, medium, and high). As a positive control (Ctrl+) hydrogen peroxide (H₂O₂) 10 mM is added, an agent which is certainly capable of causing high cell death in HL-60 cells. At t₀ the cells are labeled with a cell death marker (PI) kept in the culture medium for the entire duration of the experiment and with a fluorescent cell localization marker (7-amino-4-chloromethylcoumarin). Properties are measured at t₀ and at t₂₄.

The property used as selection property in this example was:
derived property: number of cells contained in each microwell at t₀.

The output parameter is cell mortality at t₂₄ₕ, expressed as % of dead cells, i.e., cells the intensity for which the fluorescence signal emitted by the PI marker exceeds a certain threshold.

With reference to FIG. 2, classes are selected for the selection property “number of cells per well” at t₀, in particular, classes are selected for values equal to 1, 2, 3, 4, 5, 6, 7, 8, 9, 10, 11, 12 or more than 12 cells per microwell. The data is shown on the x-axis in the graph in FIG. 2.

The output parameter, i.e., cell mortality, is extrapolated into the subsets of microwells classified as above.

It should be noted that, in the control samples, i.e., not exposed to the agent, cell death above a threshold value is measured exclusively in those subsets in which the number of cells per microwell is less than or equal to 8, as expressed by the gray scale on the Ctrl-line in the graph in FIG. 2.

The data shown in FIG. 2 indicate that, by selecting exclusively the subsets of microwells with a low basic mortality, i.e., those microwells selected to have a cell content per microwell greater than 8, the efficacy percentage of the drug is approximately equal to 80%, measured as the ratio of the percentage of dead cells in the treated sample to the control. In the subset of microwells with a content of up to 7 cells/well, i.e., those excluded from the assay due to the method according to the present invention, the percentage of efficacy would have instead been equal to about 50%, since part of the drug effect would have been masked by the presence of a higher base mortality.

The result is indicative of how the method according to the present invention allows obtaining a robust datum, excluding from the processing the subsets of microwells which would have returned an artificial datum, affected by external or environmental agents but in any case not correlated with the analysis in progress.

### Example 3: Immunotherapy Efficacy Analysis

Blood samples from individuals with multiple myeloma are made available. These samples are seeded in microwells. The selection properties used in this assay are:
direct properties: “CD38 immunophenotype”=tumor cells, “CD16-CD56 immunophenotype”=immune cells;
derived property of coexistence: co-localization of immune cells and tumor cells in the same microwell.

A subset of microwells was selected where immune system cells (NK cells) are in close proximity to CD38+ tumor cells.

The plurality of microwells was exposed to an anti-CD38 agent and the output parameter was extrapolated which is the mortality evoked by said agent measured in the selected set of areas of interest, i.e., in the tumor cells found in microwells which have co-localization with NK cells. This approach allows performing an ADCC assay (Antibody-Dependent Cellular Cytotoxicity) with high precision, i.e., limited to microwells where there is a co-localization of the two types of cells of interest.

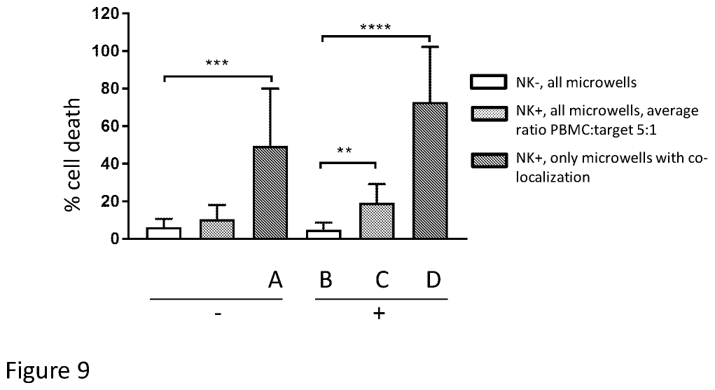

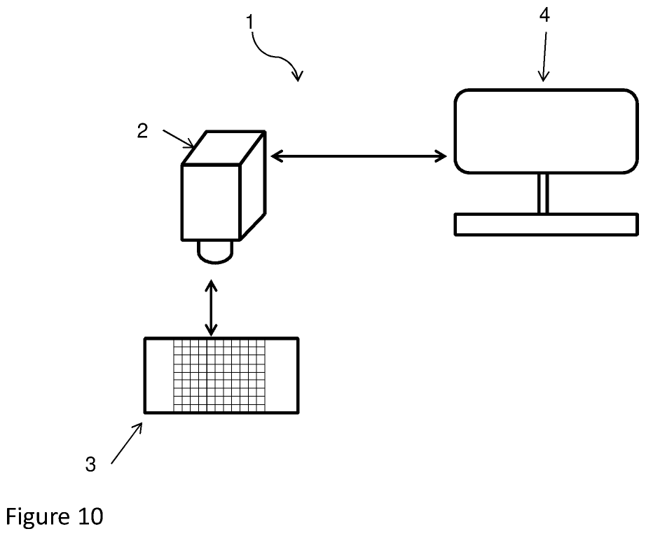

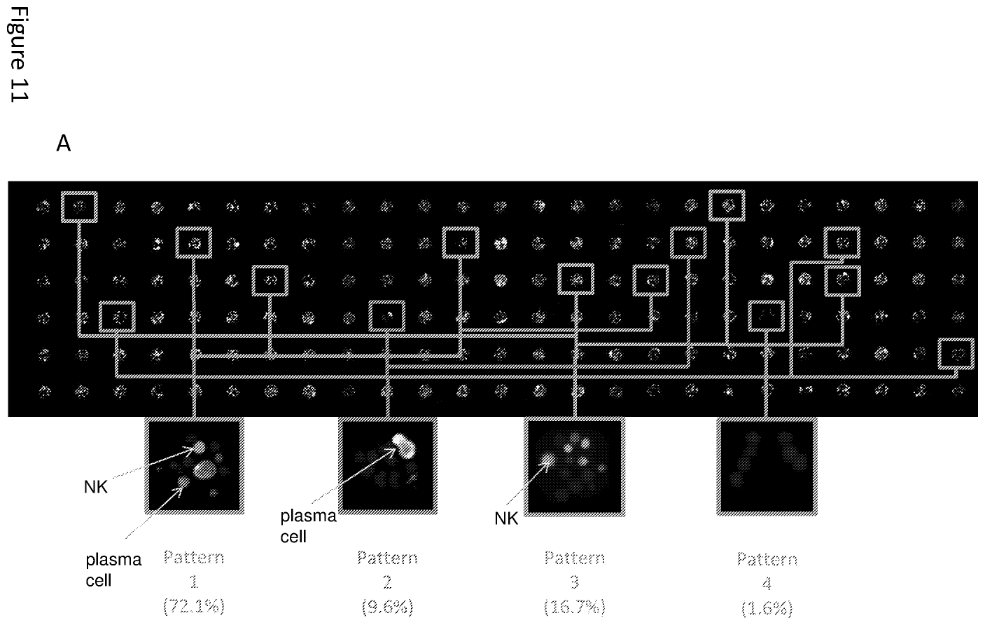

The data obtained and reported in FIG. 9 show that the activity of an anti-CD38 agent is greater in that subset of microwells which comprise immune system cells which co-localize with tumor cells (column D) compared to the average response obtained on the overall cell population (column C).

Also in this case, the removal of deviant data or which introduce noise effects into the measurement, such as wells without co-localization of the two cell types, allows achieving a more accurate measurement of the effective efficacy of the therapy and the level of activity or fitness of the patient-specific immune system cells. In the specific case, it is observed that 70% of the patient's NK cells, once stimulated with the drug, have the ability to cause cell death of the target cells placed in contact.

Further analyses and assessments on the efficacy of the drug may be conducted in the same experimental system. For example, the selection of the subset of microwells which do not comprise NK cells but only CD38+ cells, in the presence of the anti-CD38 drug, allows assessing as an output parameter the direct cytotoxic effect caused by the drug on target cells and not mediated by NK cells (column B).

By selecting subgroups of microwells which comprise CD38+ tumor cells which co-localize with NK cells, in the absence of the anti-CD38 drug it is possible to measure the spontaneous activity of NK cells towards tumor cells (column A).

Finally, further evaluations may be performed to highlight drug activity as the distance between NK cells and tumor target varies, adding the derived property “tumor cell to NK cell distance” among the selection properties.

It is worth noting that, having acquired the complete panel of properties as per panel C in FIG. 6, the assessments described herein and others which those skilled in the art will want to conduct may be carried out by independently choosing selection properties and output properties, processing the data available, as schematized in panel D in FIG. 6.

### Example 4: Control of the Co-Localization of Heterogeneous Cell Populations in the Microwells

With the aim of maximizing the probability of arranging microwells with the co-localization of at least one type A cell and one type B cell, different approaches have been defined herein, detailed below.

For the purpose of the following examples, the following definitions are assumed:

R1=the ratio of effector cells (e.g., immune system cells) to total cells in the initial cell population.

R2=the ratio of target cells (e.g., tumor cells) to total cells in the initial cell population.

E:T=the ratio of effector cells to total cells.

c=the concentration of the co-culture.

### Example 4A

On the sample isolated from the patient, divided into two tubes, a first enrichment step is carried out, obtaining in a first tube an R1 equal to about 100% and in a second tube an R2 equal to about 100%.

In doing so, it is possible to determine E:T which will be obtained by mixing together known quantities of the contents of the two tubes and it is thus possible to define c so as to obtain the desired average number of cells per microwell.

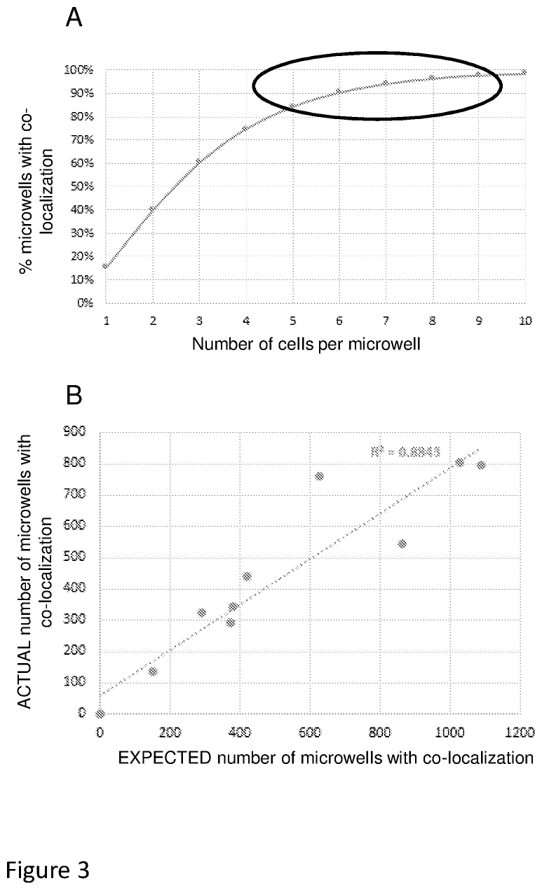

In theory, in the case of using 2 pure populations, i.e., with R1 and R2 approximately equal to 100%, by sequentially seeding the 2 populations, a co-localization probability close to 100% is obtained if an average of 10 cells/microwell is assumed (FIG. 3A, theoretical graph). The experimental data, obtained on NK cells and tumor cells enriched as described above, confirm the expected trend with good approximation (FIG. 3B, experimental data).

### Example 4B

The sample under analysis comprises effector cells in PBMCs (peripheral blood mononuclear cells) isolated from the patient at varying frequencies without any enrichment (e.g., R1=5-20% within 8 samples analyzed).

The tumor cells are enriched or, alternatively, a cell line is used (R2˜100%).

Optimal E:T is Known from Probability Theory

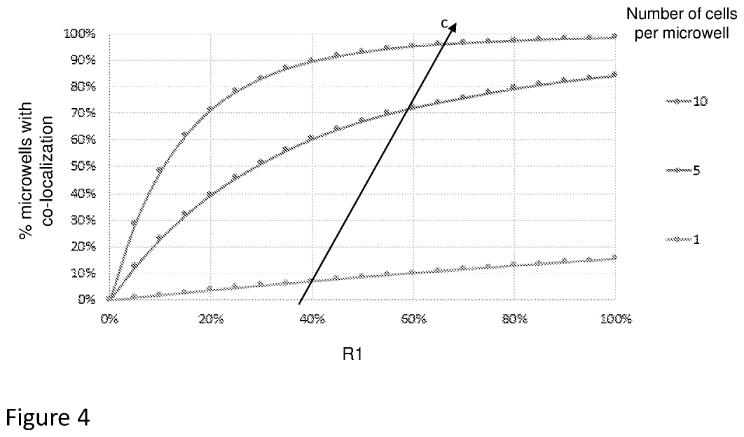

The effector cells and target cells are seeded sequentially. Assuming to have an average of 10 cells/well, the probability of effector/target cell co-localization is between 30% and 70%. In particular, as shown in the graph in FIG. 4, with R1=5 the probability of co-localization is 30%, with R1=20% the probability rises to 70%. The graph also shows that the ideal number of cells per microwell to obtain the maximum co-localization is approximately 10 cells/microwell. Having 1,200 microwells for each condition, also considering a reduction of microwells in the limit condition of 30%, a good statistical significance is maintained thanks to the replication of the microwells.

### Example 4C

For this test, NK effector cells are made available in PBMCs isolated from the patient at varying frequencies (e.g., R1=5-20%).

The tumor cells are also variable-frequency in the same patient's PBMCs.

In this case, i.e., by using a single population withdrawn from a patient containing a range between 5-20% of effector cells of interest and a variable range of tumor cells, very different situations can be obtained in terms of co-localization probability. Some examples show that the values predicted by theoretical calculations are reached with a good approximation. The minimum usable extremes of the frequency ranges of the two cell populations depend on the number of microwells available and the statistical power required.

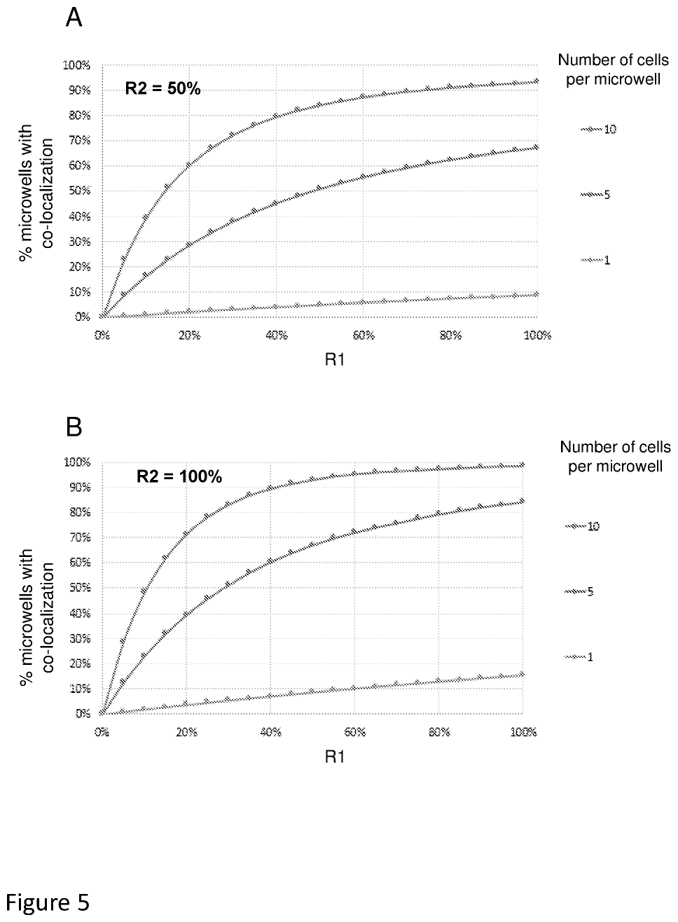

By way of example, the graph in FIG. 5 shows the co-localization frequency observed, as the number of cells/microwell varies, with R2=50% or with R2=100%.

In a real case, subject A showed R1=17.3 and R2=28.1. The theoretical calculation led to estimate a co-localization in 57.2% of the microwells. The experimental data led to observe a co-localization in 48.1% of the microwells. In a further experimental case, subject B showed R1=14.2 and R2=10.0. The theoretical calculation led to estimate a co-localization in 56% of the microwells. The experimental data led to observe a co-localization in 56.2% of the microwells.

### Example 5: Assays on Cells from Patients with Multiple Myeloma

EDTA bone marrow samples were collected from 13 patients with multiple myeloma (MM, 7 de novo and 6 relapses). 8 primary samples were processed through density centrifugation (Ficoll-Pacque; Merck) in order to obtain mononuclear cells while preserving the original composition of the effector (E) and target (T) cells, i.e., NK and plasma cells, respectively. 5 samples were processed with CD138 Antibody coupled to magnetic beads (Miltenyi Biotec) to obtain a population of white blood cells (WBC), a population which comprises NK cells and is depleted of plasma cells.

The resulting cells were co-cultured with U-266 or NCI-H929 cell lines as target cells. The U-266 cells were kept growing at 37° C. with 5% CO₂ in 1640 RPMI medium (Sigma-Aldrich) supplemented with 10% fetal bovine serum (Sigma-Aldrich), 1% L-glutamine (Sigma-Aldrich) and 1% penicillin/streptomycin mixture (Sigma-Aldrich). The NCI-H929 cells were cultured at 37° C. with 5% CO₂ in 1640 RPMI medium (Sigma-Aldrich) admixed with 20% fetal bovine serum (Sigma-Aldrich), 1% L-glutamine (Sigma-Aldrich), 1% penicillin/streptomycin mixture (Sigma-Aldrich) and 1% sodium pyruvate (Merck).

The cells from the primary samples were stained with CMAC (Thermo Fisher Scientific), used as a cell tracer. In co-culture experiments, white blood cells and target cells (U-266 or NCIH929) were stained with Calcein AM (Thermo Fisher Scientific) and CMAC, respectively. NK cells (effector cells, E) and plasma cells (target cells, T) were labeled using BV421 Mouse anti-Human CD16/CD56 (BD Biosciences) and AF647 Mouse anti-Human CD138 (BioRad) fluorescent antibodies, respectively. Propidium iodide (PI, Thermo Fisher Scientific) was used as a cytotoxicity marker.

Statistical Model for Cell Co-Localization

A statistical model was created to define the optimal experimental setup which produces the maximum number of microwells containing the desired effector/target co-localization pattern (derived selection property, of co-existence), defined by an effector/target co-localization factor E/T_{CF} which is the ratio of E to T in the same microwell.

The model takes into account four parameters which influence the E/T_{CF} factor:
1) the initial effector/target mixing ratio (E:T);
2) the overall concentration of the cells (c);

3) the ratio between the effector cells and the input cell population (R1) and 4) the ratio between the target cells and the input cell population (R2).

The parameters R1 and R2 depend only on the type of sample (e.g., cell line, patient primary sample), while E:T and c can generally be modified by the user to maximize the frequency of specific models of interest within the matrix of microwells. For experiments where both the E cells and T cells are the patient's primary sample cells, E:T cannot be modified and only c can be optimized.

Cell Seeding and Drug Exposure

The cells from primary or co-culture samples were seeded in 96-well plates, with a final concentration of 2×10⁵ cells/well, with variable E:T ratios. In addition, conditions with E:T ratios of 1:0 (effector cells only) and 0:1 (target cells only) were used as controls. Using a robotic microfluidics system, the cells were loaded into the microfluidic device and trapped in the microwells. The Daratumumab monoclonal antibody (anti-CD38) was used in 3 doses, administered through different microchannels (0.1 μg/mL, 1 μg/mL and 10 μg/mL), while a further microchannel, without drug, was used as a control. The drug was diluted in RPMI 1640 medium (Sigma-Aldrich) admixed with 10 or 20% fetal bovine serum (Sigma-Aldrich), 1% L-glutamine (Sigma-Aldrich), 1% penicillin/streptomycin mixture (Sigma-Aldrich). Each experiment was analyzed by time lapse in fluorescence microscopy for up to 12 hours.

### ICNP Image and Data Analysis

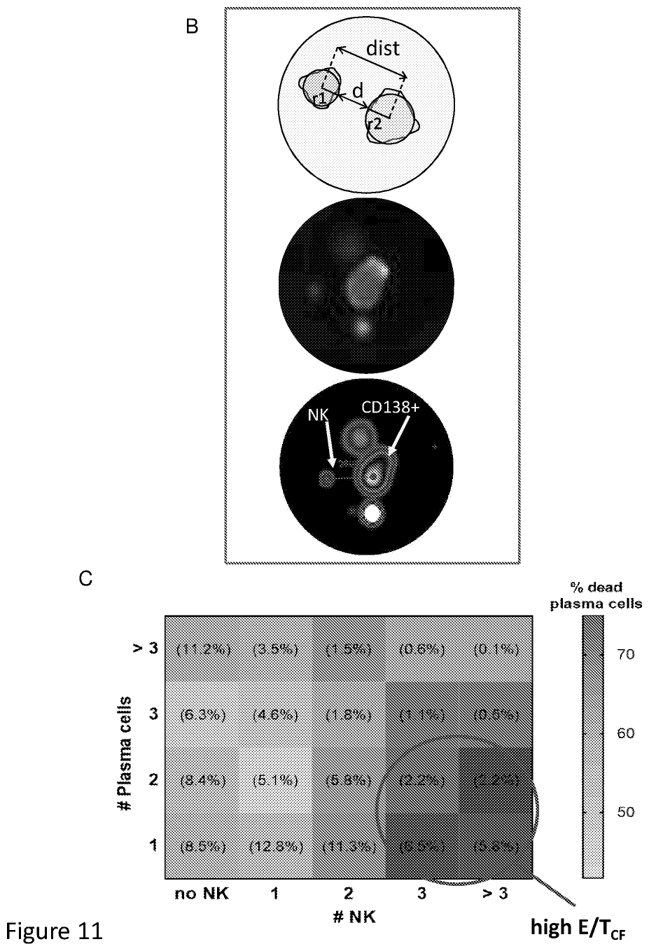

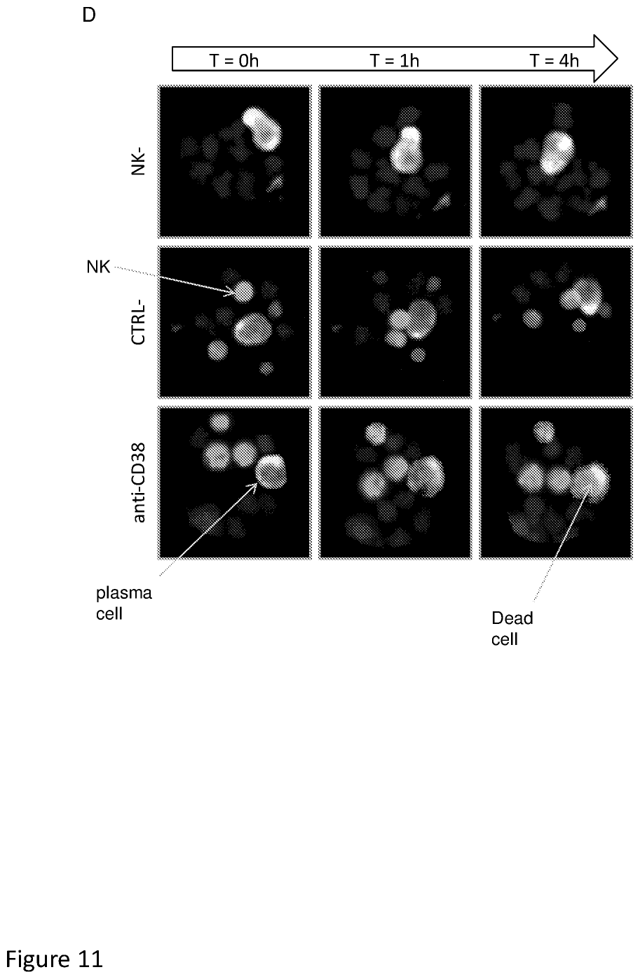

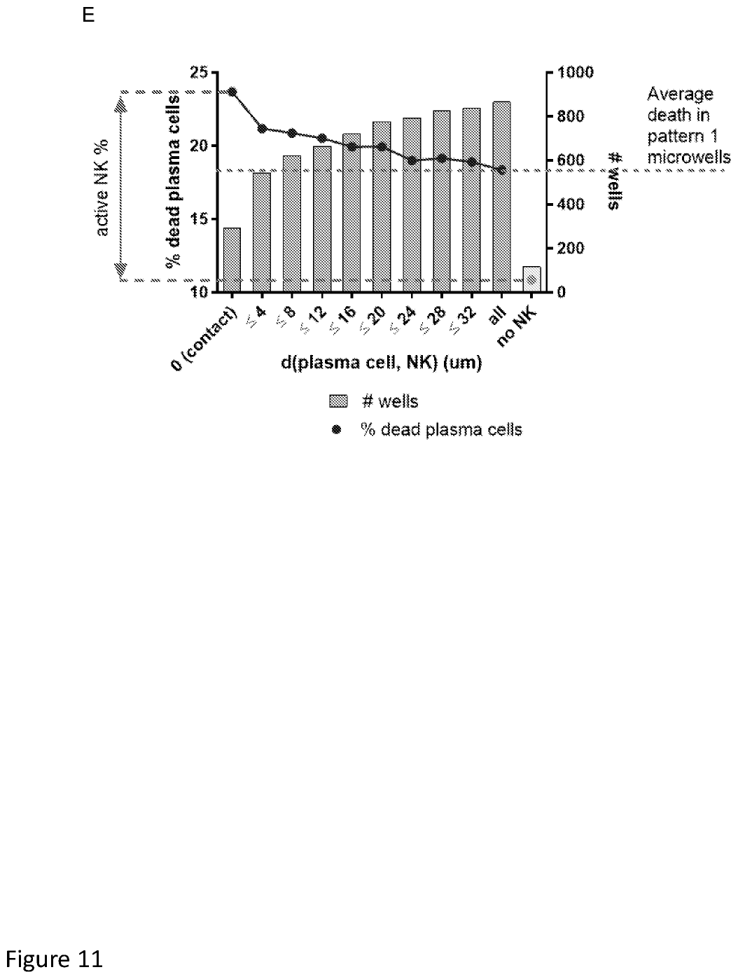

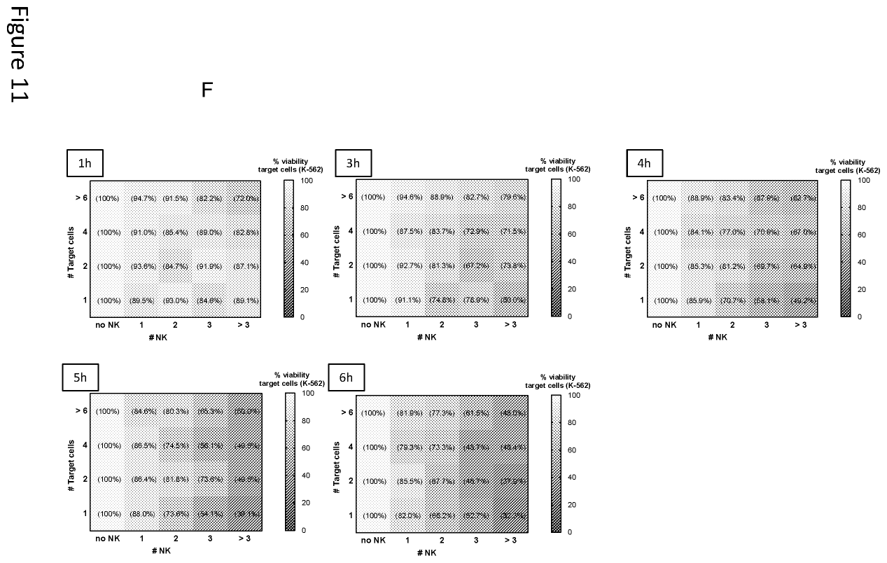

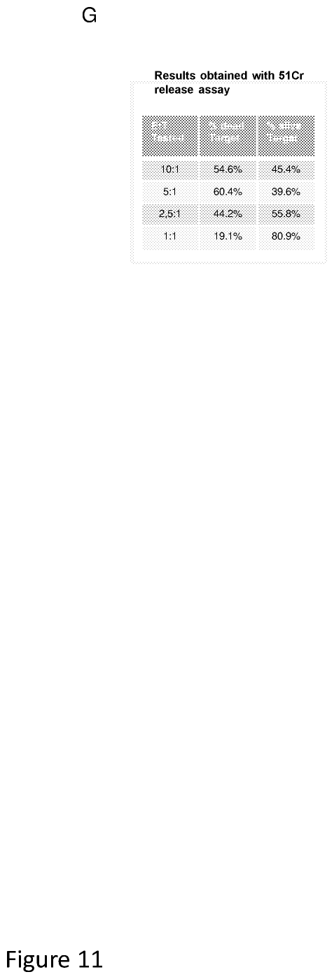

ICNP is an analytical method enabled by the availability of a large number of said microwells, based on randomly creating a huge number of heterogeneous cell clusters and then classifying and analyzing the cells into specific groups of cell clusters which share analogous cell-cell interaction patterns (FIG. 11A). The large number of clusters which are obtainable by the method according to the present invention, 19,200 in this specific example, allows identifying even relatively rare patterns or evaluating multiple interaction patterns in a single experiment while maintaining good statistical significance. In this example, the ICNP analysis was optimized to perform an ADCC assay (Antibody-Dependent Cell Cytotoxicity) for the assessment of the potency of NK cells against tumor cell lines and primary tumor cells, under stimulation with anti-CD38 (Daratumumab).

The images for said plurality of microwells are then acquired and, with a detection algorithm, the areas of interest, where each area of interest corresponds to a single cell, for each of said areas of interest, properties which comprise localization, the intensity of certain markers in each fluorescence channel, the cell area, the position of the center of gravity and the morphology are then measured. The data related to each of said properties are collected at different and subsequent times, in this case at T=0h, T=1h, T=2h, T=4h, T=12h and stored in a database.

A subset of said plurality of microwells is then selected based on said properties, where said selection is based on 4 specific co-localization patterns, as shown in FIG. 11A. Each of the patterns is characterized by E/T_{CF} values, from a specific number of E cells and a specific number of T cells. Consequently, for each channel of the microfluidic device, and on the same cell pool, multiple E/T co-localization patterns are assessed.

Furthermore, some microwells serve the function of internal control. For example, wells containing only target cells in a microchannel stimulated with a drug allow assessing the direct cytotoxicity caused by the drug.

On the subset of said plurality of microwells selected, a second classification is made, at the level of the area of interest, by evaluating the cell-cell interaction models within a specific subset of microwells, based on immunophenotype, vitality and spatial information. In this classification, a key step is the assessment of the distances and contacts between the areas of interest included in the same microwell. This information (FIG. 11B) is derived from the coordinates (x, y) of the center and radius r of each pair of areas of interest being assessed. The radius refers to a circular object having the same area as the area of interest, i.e., the single cell under analysis.

For the purpose of the method, a pair of cells is defined as “in contact” if:

d≤dist′(x1,y1),(x2,y2)/−r1−r2−tol

where dist ((x1, y1), (x2, y2)) is the distance d between the two centers, r1 and r2 are the radii of the two areas of interest and tol is a tolerance value, set at 4 μm here. For example, the target cells are classified based on the distance from the immune cells in the same microwell, thus allowing the identification of those target cells which are in contact with immune cells or those target cells which are located within a certain distance from an effector cell.

The method allowed assessing how the potency of NK cells (i.e., the cell-mediated cytotoxicity caused on the tumor cells) changes with the distance from the CD138+ cells.

Specifically, the 4 selected patterns, shown in FIG. 11A, were: pattern 1) microwells comprising NK and plasma cells (72.1%), pattern 2) only plasma cells (9.6%), pattern 3) only NK cells (16.7%), pattern 4) no cells of interest (1.6%).

The selection of said subset of microwells advantageously allowed a targeted study of NK-mediated cytotoxicity, where the study was carried out exclusively on the subset of microwells selected for pattern 1. Furthermore, a key advantage of the method according to the present invention lies in the possibility of assessing, for a certain experiment, specific co-localization patterns.

FIG. 11C shows a heatmap resulting from the analysis of an experiment in which 20 different co-localization patterns of NK and U-266 cells were analyzed, each box of the heatmap is related to a pattern. The cells were exposed to anti-CD38 antibody and each pattern differs in the number of E (NK) cells and T cells (U-266 cells) included in the same microwell, thus allowing the influence of the effector: target ratio on the death of the target cells to be assessed. The plasma cell death rate assessed in the microwells with the method according to the present invention revealed that target cell death is higher in the microwell subset with a higher E/T_{CF} ratio. The datum can be superimposed on the datum obtained with methods known in the art, i.e., in culture plates, as shown by the comparative data obtained with the Cr51 release assay (FIG. 11G), with the key advantage of being capable of measuring multiple patterns simultaneously and with a resolution of a single area of interest.

After the classification of the microwell subsets, a detailed analysis at the single cell level was performed on images acquired in time lapse. The method according to the present invention allowed investigating the effects of cellular “networking,” grouping the data by homogeneous interaction pattern. FIG. 11D shows an example of images analyzed to investigate the interaction between NK cells and plasma cells in detail. Each line in the image corresponds to a different condition: direct effect of the anti-CD38 on a target cell belonging to a microwell with pattern 2, i.e., without effector cells (NK-); effect of the spontaneous interaction between a target cell and the effector in microwells with pattern 1, without anti-CD38 stimulation (CTRL-), or with anti-CD38 stimulation with contact between NK and plasma cells (anti-CD38). The samples with pattern 1 (anti-CD38) show that the interaction causes the death of plasma cells, as detected by the absorption of propidium iodide and the consequent appearance, starting from 1 h, more evident at 2 h, of the signal (indicated with the arrow in the image). The plasma cell, on the other hand, does not die in the representative image shown for pattern 2, i.e., in the absence of effector cells. The plasma cell death in the pattern 1 microwells was assessed with respect to the distance from an NK cell, with the aim of estimating the actual potency of the NK cell which is responsible for the observed toxicity.

FIG. 11E shows the data collected from 1,200 microwells in which the cells were stimulated with Daratumumab at a dose of 10 μg/mL. The method according to the present invention allowed observing that the death is maximum for those plasma cells in contact with NK cells and decreases as the plasma cell—NK cell distance increases. The plasma cells not in direct contact but in the immediate vicinity of NK cells show an increased mortality rate compared to the cells further away. These data are indicative of the fact that the activation of an NK cell not only impacts the cell with which it comes into direct contact but can also affect the surrounding environment, i.e., the cells located at a minimum distance from the NK cell which are likely to be subjected to death caused by a contact with the NK cell which can occur in a different instant of time than that corresponding to the observation or due to the secretion of toxic substances such as perforin and granzymes following the first activation of the NK cell from contact. Hence the value of the method according to the present invention, which even during the analysis on a single cell takes into consideration the environment in which said cell is contained, thus allowing the selection of representative subsets of the environment of interest.

In the experiment reported, the method allowed estimating that the fraction of powerful NK cells, i.e., capable of killing the target when contact is provided, is 12.82% of the total. This number was calculated as the difference between the mortality rate of plasma cells belonging to pattern 1, therefore in contact with an NK cell (23.68%) and the death rate of the plasma cells belonging to pattern 2 (10.86%), which is due to spontaneous death or a direct effect of the anti-CD38 antibody. The heatmap in FIG. 11F shows the results of cell viability measured in the different patterns over time.

1-15. (canceled)

16. A method for subjecting a plurality of microwells containing cells to a high-content assay, said method comprising:
(a) acquiring at least one image of said plurality of microwells;
(b) in said image, detecting a plurality of areas of interest, each area of interest corresponding to a single cell;
(c) measuring at least one derived property, and, optionally, at least one direct property of said areas of interest, wherein said derived property is a property associated with a multiplicity of areas of interest, i.e., a property which requires the assessment of two or more areas of interest to be measured, wherein one of said derived properties is a relationship property, or is a coexistence property, between one or more areas of interest included in the same microwell, and said direct property is a property associated with a single area of interest, i.e., a property measured by assessing the single area of interest where said one or more properties is a selection property;
(d) selecting a subset of said plurality of microwells, where said microwells belonging to the subset contain areas of interest selected based on said at least one selection property; and
(e) extrapolating an output parameter from a property measured in the set of areas of interest selected, where said property is defined as output property, said output property being distinct from said selection properties where said output parameter is the processing of an output property measured in said set of areas of interest.

17. A method according to claim 16, wherein said selection is made by imposing inclusion criteria, wherein said inclusion criteria comprise:
(1) identifying, from said derived and, optionally, direct measured properties, one or more selection properties; and
(ii) imposing, for each of said selection properties, the threshold value, or the range of values, within which said selection property must fall.

18. A method according to claim 16, wherein at least one of said selection properties is a cumulative property.

19. A method according to claim 16, which comprises the selection of a first set of areas of interest based on a first selection property and, within said first set of areas of interest, a selection of a subset of areas of interest based on a second selection property, preferably, said first selection property is a cumulative property and said first set of areas of interest corresponds to a subset of microwells and said second selection property is a direct or relative property, and said subset of areas of interest corresponds to a subset of cells embedded in said subset of microwells.

20. A method according to claim 16, wherein said at least one derived property is the co-localization of at least two cells with different immunophenotypes in the same microwell.

21. A method according to claim 20, wherein said at least two cells with different immunophenotypes are immune cells and tumor cells.

22. A method according to claim 16, wherein said at least one derived property is the average distance of each cell from the other cells belonging to the same microwell.

23. A method according to claim 16, wherein said output parameter is the result of any statistical processing of the output property measured in each area of interest which belongs to the set of areas of interest selected.

24. A method according to claim 16 wherein said at least one image is acquired with an image acquisition device configured to acquire at least one image of said plurality of microwells.

25. A method according to claim 16, wherein said image is analyzed and processed to return a measurement of said properties, said analysis and processing process comprising the following steps:
(1) identifying, in an image containing a plurality of microwells, the zones which correspond to the microwells;
(2) detecting, within said zones corresponding to the microwells, a plurality of areas of interest, each area of interest corresponding to one of said cells contained in said plurality of microwells;
(3) measuring at least one property of each of said areas of interest;
(4) selecting a set of areas of interest based on one or more of said measured properties, said one or more properties defined as selection properties; and
(5) extrapolating an output parameter from measured properties in said set of areas of interest.

26. A method according to claim 16, wherein said plurality of microwells is embedded in a microfluidic device comprising at least 15,000, or at least 18,000, preferably 19,200 microwells.

27. A method according to claim 16, wherein said microwells are inverted open microwells, i.e., are microwells which have an upper end and a lower end both open.

28. A method according to claim 16, which subjects said plurality of microwells to a dynamic test, where several images of the same field are acquired at successive times (time-lapse imaging) and the measurement of said at least one property, at time t₀ and, subsequently, at time t₁, t₂, . . . tₙ, returns an analysis which reflects the variations of said property over time.

29. A method according to claim 16, wherein said cells, while kept in said plurality of microwells, are exposed to one or more agents which impact said output parameter.

30. A system (1) for subjecting a plurality of microwells containing cells to a high-content assay, said system comprising:
(a′) an image acquisition device (2) configured to acquire at least one image of said plurality of microwells (3); and
(b′) a data processing unit (4) configured to:
(1′) in said image, detecting a plurality of areas of interest, each area of interest corresponding to a single cell;
(2′) measure at least one derived property, and, optionally, at least one direct property of said areas of interest, wherein said direct property is a property associated with a single area of interest, i.e., a property measured by assessing the single area of interest and said derived property is a property associated with a multiplicity of areas of interest, i.e., a property which requires the assessment of two or more areas of interest to be measured, said derived properties being a relationship property, or is a coexistence property, between one or more areas of interest included in the same microwell, where said one or more properties is a selection property;
(3′) select a subset of said plurality of microwells, where said microwells belonging to the subset contain areas of interest selected based on said at least one selection property; and
(4′) extrapolate an output parameter from a property measured in the set of areas of interest selected, where said property is defined as output property, said output property being distinct from said selection properties where said output parameter is the processing of an output property measured in said set of areas of interest.

31. A computer program for subjecting a plurality of microwells containing cells to a high-content assay, said computer program comprising instructions which, when the program is executed by a data processing unit, cause the processing unit to perform the following steps:
(a) measuring at least one derived property, and, optionally, at least one direct property of said areas of interest, wherein said direct property is a property associated with a single area of interest, i.e., a property measured by assessing the single area of interest and said derived property is a property associated with a multiplicity of areas of interest, i.e., a property which requires the assessment of two or more areas of interest to be measured, said derived properties being a relationship property, or is a coexistence property, between one or more areas of interest included in the same microwell, where said one or more properties is a selection property;
(b) selecting a subset of said plurality of microwells, where said microwells belonging to the subset contain areas of interest selected based on said at least one selection property; and
(c) extrapolating an output parameter from a property measured in the set of areas of interest selected, where said property is defined as output property, said output property being distinct from said selection properties where said output parameter is the processing of an output property measured in said set of areas of interest.
